(19) United States
(12) Patent Application Publication
(10) Pub. No.: US 2022/0323656 A1
(43) Pub. Date: Oct. 13, 2022

(54) PORTABLE BREAST PUMP

(71) Applicant: Ying Hong, Wuxi (CN)

(72) Inventor: Ying Hong, Wuxi (CN)

(21) Appl. No.: 17/851,093

(22) Filed: Jun. 28, 2022

(30) Foreign Application Priority Data
Sep. 22, 2021 (CN) 202122301565.1

(51) Int. Cl. A61M 1/06

(52) U.S. Cl. CPC A61M 1/062; A61M 2209/088

(57) ABSTRACT

The present disclosure provides a portable breast pump. The existing technical problems are solved. The portable breast pump includes a pump seat; the pump seat is hollowed, and a milk collection cavity is formed in the middle; a liquid inlet hole communicated with the milk collection cavity is formed in one side of the pump seat in a vertical direction; and a bottom side of the pump seat is provided with a planar bottom surface for placement or a bottom surface for placement sunken towards the milk collection cavity. The present disclosure has the advantages of convenience in carrying and placement, and the like.

### TECHNICAL FIELD

The present disclosure belongs to the technical field of mother and baby products, and particularly relates to a portable breast pump.

### BACKGROUND

A breast pump is well-known equipment for extracting breast milk from user's breasts. A breast pump may be used if a baby or an infant is not able to suck the breasts for breast milk by herself/himself, or if the mother is not beside the baby or infant, for example, if the mother is not beside the baby during working. When used to draw breast milk, the breast pump can also be used to stimulate galactopoiesis and to increase the amount of milk of a woman with less milk. The breast pump uses vacuum to draw breast milk from the breasts of a nursing mother. However, the existing breast pump is complicated in structure and inconvenient to carry.

In response to this problem, people have also carried out exploration and research in long-term production and life practice. For example, Chinese utility model patent discloses a gynecological breast pump [application number: 201920412434.0]. The utility model patent includes a bottle body; a bottle neck is in threaded connection to a top of the bottle body; a top of the bottle neck is in clamping connection with a pressing block; and one side of the bottle neck is in clamping connection with a breast pump head. In a use process of this utility model, when the breast pump is used to cooperate with the pressing block to draw breast milk, a clamping block which is originally placed on one side of the breast pump is clamped into the breast pump, and a groove is separated from a clamping cover; lighter first-stage breast milk can enter the collection bottle through a flowing pipe for collection. After the first breast milk is completely collected, the groove is clamped into the clamping cover, and the clamping block is taken out, so that nutrient second-stage breast milk can be normally collected into the bottle body.

The above solution improves some of the problems in the prior art to a certain extent, but the solution still has at least the following defects: complex structure and inconvenience of carrying.

### SUMMARY

The present disclosure aims to provide a portable milk pump convenient to carry for the above problems.

In order to achieve the above objective, the present disclosure adopts the following technical solution: The portable breast pump includes a pump seat; the pump seat is hollowed, and a milk collection cavity is formed in the middle; a liquid inlet hole communicated with the milk collection cavity is formed in one side of the pump seat in a vertical direction; and a bottom side of the pump seat is provided with a planar bottom surface for placement or a bottom surface for placement sunken towards the milk collection cavity.

According to the breast pump, a milk sucking-out function and a milk collection function are directly integrated together through the pump seat with the milk collection cavity, so that the breast pump is convenient to carry. During use, the breast is placed at the liquid inlet hole; the pump seat is pressed to generate a negative pressure in the milk collection cavity to suck out breast milk, and the breast milk is directly stored in the milk collection cavity; after the sucking out of the breast milk is completed, the breast pump can be directly placed and stored on a tabletop through the bottom surface for placement; and therefore, the structure is simple, and the use is flexible.

In the above-mentioned portable breast pump, the pump seat includes a skin-friendly end surface and an accommodating seat; the skin-friendly end surface and the accommodating seat are of an integrated structure; the accommodating seat is made of a silica gel material; the skin-friendly end surface is made of a silica gel or hard material; and the liquid inlet hole is formed in the skin-friendly end surface. The integrated structure of the skin-friendly end surface and the accommodating seat makes the structure of the breast pump more reliable. The accommodating seat is made of the silica gel material. It is more labor-saving when the negative pressure in the milk collection cavity is generated in a pressing way.

In the above-mentioned portable breast pump, the skin-friendly end surface is also provided with a binding surface sunken towards the milk collection cavity. The binding surface here is sunken towards the milk collection cavity can cause the skin-friendly end surface to be more in line with the surface of the breast, thus improving the air tightness during suction of the breast milk.

In the above-mentioned portable breast pump, the binding surface is made of a transparent material. The binding surface is transparent to facilitate observing the milk storage volume in the milk collection cavity.

In the above-mentioned portable breast pump, the binding surface is also provided with volume markers located below the liquid inlet hole. The volume markers are configured for quantifying the breast milk.

In the above-mentioned portable breast pump, a top side of the accommodating seat upwardly extends to form a pressure relief seat with a pressure relief hole; and the pressure relief hole is communicated with the milk collection cavity. The pressure relief hole here is used for relieving the pressure of the milk collection cavity.

In the above-mentioned portable breast pump, a top side of the skin-friendly end surface upwardly extends to form a handheld part; a plane where a top side of the handheld part is located is higher than a plane where an upper end surface of the accommodating seat is located; and a reinforcement structure is also arranged between the handheld part and the accommodating seat. The handheld part is configured for carrying the breast pump.

In the above-mentioned portable breast pump, the reinforcement structure includes a reinforcement rib arranged between a side part of the handheld part (and a top side of the accommodating seat; and the reinforcement rib is solid.

In the above-mentioned portable breast pump, the reinforcement rib, the handheld part and the accommodating seat are of an integrated structure. The handheld part and the accommodating seat can be connected into a whole through the reinforcement rib, which can avoid increase of the rigidity of the handheld part and prevent the phenomenon that folding the handheld part during carrying causes the breast milk in the milk collection cavity to shake and be poured out.

In the above-mentioned portable breast pump, an outer circumferential edge of the accommodating seat is arc-shaped. It is easier to press the accommodating seat.

Compared with the prior art, the portable breast pump has the advantages that the breast pump has the milk sucking-out function and the milk collection function, and is simple in structure and convenient to carry. After the sucking out of breast milk is completed, the breast pump can be directly placed and stored on a tabletop through a bottom surface for placement, so that it is used flexibly.

### BRIEF DESCRIPTION OF THE DRAWINGS

FIG. 1 is a schematic structural diagram provided in the present disclosure; and

FIG. 2 is a schematic structural diagram of the other side provided in the present disclosure.

In the drawings, pump seat 1, milk collection cavity 2, liquid inlet hole 3, bottom surface 4 for placement, skin-friendly end surface 5, accommodating seat 6, binding surface 7, volume marker 8, pressure relief hole 9, pressure relief seat 10, handheld part 11, reinforcement structure 12, and reinforcement rib 13.

### DETAILED DESCRIPTION OF THE EMBODIMENTS

The present disclosure is further described in detail below in combination with the accompanying drawings and specific implementations.

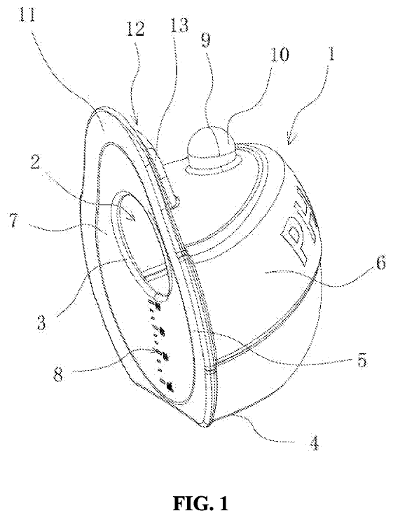

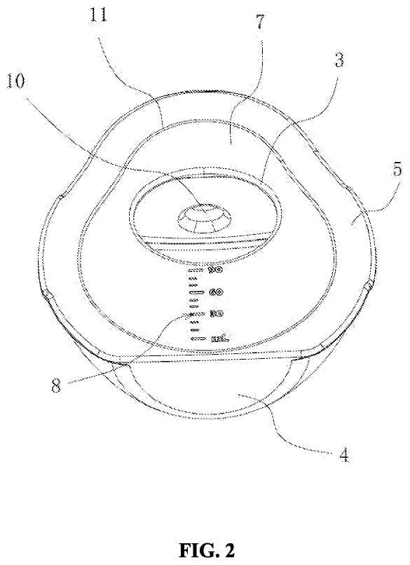

As shown in FIGS. 1-2, a portable breast pump includes a pump seat 1. The pump seat 1 is hollowed, and a milk collection cavity 2 is formed in the middle; a liquid inlet hole 3 communicated with the milk collection cavity 2 is formed in one side of the pump seat 1 in a vertical direction; and a bottom side of the pump seat 1 is provided with a planar bottom surface 4 for placement or a bottom surface 4 for placement sunken towards the milk collection cavity 2.

According to the breast pump, a milk sucking-out function and a milk collection function are directly integrated together through the pump seat 1 with the milk collection cavity 2, so that the breast pump is convenient to carry. During use, the breast is placed at the liquid inlet hole 3; the pump seat 1 is pressed to generate a negative pressure in the milk collection cavity 2 to suck out breast milk, and the breast milk is directly stored in the milk collection cavity 2; after the sucking out of the breast milk is completed, the breast pump can be directly placed and stored on a tabletop through the bottom surface 4 for placement; and therefore, the structure is simple, and the use is flexible.

Preferably, the pump seat 1 includes a skin-friendly end surface 5 and an accommodating seat 6; the skin-friendly end surface 5 and the accommodating seat 6 are of an integrated structure; the accommodating seat 6 is made of a silica gel material; the skin-friendly end surface 5 is made of a silica gel or hard material; and the liquid inlet hole 3 is formed in the skin-friendly end surface 5. The integrated structure of the skin-friendly end surface 5 and the accommodating seat 6 makes the structure of the breast pump more reliable. The accommodating seat 6 is made of the silica gel material. It is more labor-saving when the negative pressure in the milk collection cavity 2 is generated in a pressing way.

Further, the skin-friendly end surface 5 is also provided with a binding surface 7 sunken towards the milk collection cavity 2. The binding surface here is sunken towards the milk collection cavity 2 can cause the skin-friendly end surface 5 to be more in line with the surface of the breast, thus improving the air tightness during suction of the breast milk. The binding surface 7 is made of a transparent material. The binding surface 7 is transparent to facilitate observing the milk storage volume in the milk collection cavity 2. The binding surface 7 is also provided with volume markers 8 located below the liquid inlet hole 3. The volume markers 8 are configured for quantifying the breast milk.

Much further, a top side of the accommodating seat 6 upwardly extends to form a pressure relief seat 10 with a pressure relief hole 9; and the pressure relief hole 9 is communicated with the milk collection cavity 2. The pressure relief hole 9 here is used for relieving the pressure of the milk collection cavity. A top side of the skin-friendly end surface 5 upwardly extends to form a handheld part 11; a plane where a top side of the handheld part 11 is located is higher than a plane where an upper end surface of the accommodating seat 6 is located; and a reinforcement structure 12 is also arranged between the handheld part 11 and the accommodating seat 6. The handheld part 11 is configured for carrying the breast pump.

The reinforcement structure 12 includes a reinforcement rib 13 arranged between a side part of the handheld part 11 and a top side of the accommodating seat 6; and the reinforcement rib 13 is solid.

In addition, the reinforcement rib 13, the handheld part 11 and the accommodating seat 6 are of an integrated structure. The handheld part 11 and the accommodating seat 6 can be connected into a whole through the reinforcement rib 13, which can avoid increase of the rigidity of the handheld part 11 and prevent the phenomenon that folding the handheld part 11 during carrying causes the breast milk in the milk collection cavity 2 to shake and be poured out. An outer circumferential edge of the accommodating seat 6 is arc-shaped, so that it is easier to press the accommodating seat 6.

An outer edge of the accommodating seat 6 is also provided with a reinforcement bar. By means of the reinforcement bar, the stress intensity that the accommodating seat 6 can bear can be increased, thus preventing excessive deformation caused by the fact that the accommodating seat 6 is flattened.

The specific embodiments described herein are only for illustration of the spirit of the present disclosure. Those skilled in the art of the present disclosure can make a variety of modifications or supplementations to the described specific embodiments, or substitute the described specific embodiments by similar methods without departing from the spirit of the present disclosure or falling beyond the scope defined by the attached claims.

The terms, such as pump seat 1, milk collection cavity 2, liquid inlet hole 3, bottom surface 4 for placement, skin-friendly end surface 5, accommodating seat 6, binding surface 7, volume marker 8, pressure relief hole 9, pressure relief seat 10, handheld part 11, reinforcement structure 12, and reinforcement rib 13, are more frequently used herein, but the possibility of using other terms is not excluded. These terms are used only to more conveniently describe and explain the essence of the present disclosure; and it is contrary to the spirit of the present disclosure to interpret them as any kind of additional limitations.

What is claimed is:

1. A portable breast pump, comprising a pump seat (1), wherein the pump seat (1) is hollowed, and a milk collection cavity (2) is formed in the middle; a liquid inlet hole (3) communicated with the milk collection cavity (2) is formed in one side of the pump seat (1) in a vertical direction; and a bottom side of the pump seat (1) is provided with a planar bottom surface (4) for placement or a bottom surface (4) for placement sunken towards the milk collection cavity (2).

2. The portable breast pump according to claim 1, wherein the pump seat (1) comprises a skin-friendly end surface (5) and an accommodating seat (6); the skin-friendly end surface (5) and the accommodating seat (6) are of an integrated structure; the accommodating seat (6) is made of a silica gel material; the skin-friendly end surface (5) is made of a silica gel or hard material; and the liquid inlet hole (3) is formed in the skin-friendly end surface (5).

3. The portable breast pump according to claim 2, wherein the skin-friendly end surface (5) is also provided with a binding surface (7) sunken towards the milk collection cavity (2).

4. The portable breast pump according to claim 3, wherein the binding surface (7) is made of a transparent material.

5. The portable breast pump according to claim 4, wherein the binding surface (7) is also provided with volume markers (8) located below the liquid inlet hole (3).

6. The portable breast pump according to claim 2, wherein a top side of the accommodating seat (6) upwardly extends to form a pressure relief seat (10) with a pressure relief hole (9); and the pressure relief hole (9) is communicated with the milk collection cavity (2).

7. The portable breast pump according to claim 2, wherein a top side of the skin-friendly end surface (5) upwardly extends to form a handheld part (11); a plane where a top side of the handheld part (11) is located is higher than a plane where an upper end surface of the accommodating seat (6) is located; and a reinforcement structure (12) is also arranged between the handheld part (11) and the accommodating seat (6).

8. The portable breast pump according to claim 7, wherein the reinforcement structure (12) comprises a reinforcement rib (13) arranged between a side part of the handheld part (11) and a top side of the accommodating seat (6); and the reinforcement rib (13) is solid.

9. The portable breast pump according to claim 8, wherein the reinforcement rib (13), the handheld part (11) and the accommodating seat (6) are of an integrated structure.

10. The portable breast pump according to claim 2, wherein an outer circumferential edge of the accommodating seat (6) is arc-shaped.
